(19) United States
(12) Patent Application Publication
(10) Pub. No.: US 2026/0224522 A1
(43) Pub. Date: Aug. 6, 2026

(54) AN IMMEDIATE-RELEASE ORAL PHARMACEUTICAL FORM OF ESCITALOPRAM OR RACEMATE THEREOF WITH INCREASED API CONTENT

(71) Applicant: Lasse KINAST, Berlin (DE)

(72) Inventor: Lasse KINAST, Berlin (DE)

(73) Assignee: Lasse KINAST, Berlin (DE)

(21) Appl. No.: 19/154,095

(22) PCT Filed: Feb. 7, 2024
(86) PCT No.: PCT/EP2024/053066

§ 371 Date: Aug. 6, 2025

(30) Foreign Application Priority Data
Feb. 7, 2023 (DE) 10 2023 000 367.0

(51) Int. Cl. A61K 31/343; A61K 9/20; A61K 9/28

(52) U.S. Cl. CPC A61K 31/343; A61K 9/2013; A61K 9/2054; A61K 9/2095; A61K 9/2893

(57) ABSTRACT

An immediate-release oral pharmaceutical composition is provided which comprises by weight either ≥60.1% Citalopram Oxalate or Escitalopram Oxalate or ≥25.1% Citalopram Oxalate or Escitalopram Oxalate at a median particle size of more than 40 μm, and from 0% to 74.9% additional pharmaceutical excipient. Escitalopram and the racemate thereof (Citalopram) are used to treat depression and generalized anxiety disorder (GAD). They belong to a group of medicines known as selective serotonin reuptake inhibitors (SSRIs).

The invention provides the composition of an immediate-release oral pharmaceutical form which comprises by weight either ≥60.1% Escitalopram Oxalate or ≥25.1% Escitalopram Oxalate (at a median particle size of more than 40 μm), and from 0% to 74.9% additional pharmaceutical excipient. With the composition invented, the prepared Escitalopram Oxalate immediate-release oral pharmaceutical form has the benefit of a higher percentage API content and a lowered percentage excipient content, while keeping with the requirements to the pharmaceutical quality. The Escitalopram Oxalate entails also the other pharmaceutically acceptable salts of Escitalopram and the racemate (Citalopram) and pharmaceutically acceptable salts thereof.

### TECHNICAL FIELD

The invention belongs to the field of pharmaceutical preparations, a composition of matter, for the composition of an Escitalopram, or racemate thereof, immediate-release oral pharmaceutical form.

### BACKGROUND

Escitalopram is an antidepressant of the selective serotonin reuptake inhibitor (SSRI) class.

Escitalopram and the racemate thereof (Citalopram) are used to treat depression and generalized anxiety disorder (GAD). They are antidepressants that belong to a group of medicines known as selective serotonin reuptake inhibitors (SSRIs). These medicines work by increasing the activity of the chemical serotonin in the brain.

Escitalopram is typically administered in the form of Escitalopram Oxalate as immediate release film-coated tablets, (e.g., Cipralex) or controlled-release tablets. A typical Escitalopram film-coated tablet on the market contains roughly 11% Escitalopram Oxalate. The standard dosages being 5 mg, 10 mg, 15 mg and 20 mg, and the typical dosage form being film-coated tablet. Escitalopram film-coated tablets are usually and out of necessity packaged in PVC/ALU or ALU/ALU blisters, to ensure the stability of the product.

Citalopram is typically administered in the form of Escitalopram Hydrobromide as immediate release film-coated tablets, (e.g., Cipramil). A typical Citalopram film-coated tablet on the market contains roughly 20% Citalopram Hydrobromide. The standard dosages being 10 mg, 20 mg, 30 mg and 40 mg. Citalopram film-coated tablets are usually and out of necessity packaged in PVC/ALU or ALU/ALU blisters, to ensure the stability of the product.

The environmental burden of pharmaceutical packaging is immense. This is because the aluminium and plastics used to form the blister packaging are difficult and even impossible to recycle effectively as the materials are sealed together. Separating the materials again requires high amounts of energy and even then, the aluminium received from this is not of the same high quality as new aluminium. The larger the immediate-release oral pharmaceutical dosage form is, the larger the primary packaging must be and therefore also the more waste material is created.

Additionally, there is the environmental burden caused through the extensive transportation steps required of the finished product, such as the transportation from the manufacturer to the wholesaler and then to the pharmacy. Each box of finished product in the pharmaceutical industry needs to be transported several steps until it reaches the customer, often even global transportation routes are involved. This is especially the case for generic medicines, and further relevant for products which are sold in large quantities, like with Escitalopram. The volume of the finished product determines how efficiently transportation and storage can be conducted, since a product with a high volume will require more space and more shipments compared to the same product at a smaller volume.

Furthermore, when it is avoidable to expose the patient to excipients, it should be done. An immediate-release oral pharmaceutical form is administered with the goal of subjecting the human body to the drug and not the excipients. It is a waste of resources of the pharma industry to use excipients in products where they can be avoided, especially when used in high quantities. Also, the global demand of excipients is ever increasing, and the supply capabilities of the chemical industry is falling behind the demand which makes this waste of recourses even more problematic.

Previous patented combinations of Escitalopram tablets, such as WO2009150665 and WO2018190294 reach an Escitalopram content by weight of 25.0% and 10.2% respectively. The highest Escitalopram content of 60.0% has been found possible in the patent EP2086509 with the stipulation, that the median particle size of Escitalopram must be less than 40 μm. Citalopram, the racemate of Escitalopram, has been implemented in contents of 60.0% in EP1318805A2, again with the stipulation that the median particle size of Citalopram must be less than 40 μm for this to function. A 2014 Dissertation from Aravapalli V. S. A. Kumar (Aravapalli, V. S. A. Formulation and Evaluation of Escitalopram Oxalate Immediate Release Tablets. 2014. Dissertation. College of Pharmacy, Jaya College of Paramedical Sciences, Chennai) describes a compatibility study on escitalopram oxalate, in which powder mixtures of the API, possibly with excipients, were filled into vials and then analysed in excipient compatibility studies. The preparation of 20 mg escitalopram tablets is also described, but these have different compositions from the above-mentioned powder mixtures and do not contain increased concentrations of the active ingredient. Tablets or capsules with increased active substance concentrations are not described.

We have surprisingly found a way to create an immediate-release oral pharmaceutical form of Escitalopram with an increased percentage Escitalopram content and have also surprisingly found that the particle size restriction, previously thought necessary to reach a high percentage content of Escitalopram, is in fact not needed. The invention shows that the percentage content of Escitalopram can be implemented above 25.0% with median size particles >40 μm, and that the percentage content of Escitalopram can be brought to above 60.0%.

Currently, on the market Escitalopram 5 mg film-coated tablets weigh roughly 60 mg, and Escitalopram 20 mg film-coated tablets weigh roughly 240 mg. The excipient content is 54 mg and 214 mg per film-coated tablet respectively. Through this invention the excipient content can be brough down to 0%, in the form of a tablet, or roughly 3% when accounting for the film-coating of a film-coated tablets.

As the amount of excipient needed is reduced to a minimum, the immediate-release oral pharmaceutical form shrinks, which in turn decreases the size of the blister and therefore decreases the size of the entire finished product. Now a previously impossibly small sized blister can be used, and this enables the finished product to have a fraction of the current volume. This in turn allows for much more efficient transportation, lowering the carbon footprint of the product, and only a fraction of the packaging waste is created.

It also reduces the amount of excipient administered to the patient down to the indispensable and drastically alleviates the pressure on the pharmaceutical industry to seek out and process an unnecessarily high quantity of excipient.

As Escitalopram is a high-volume prescription medication, it is synthesized in many production plants and the finished product is manufactured globally. The facilities and equipment needed to implement production of the given combination is readily available. The following examples of composition and preparation can realistically be implemented at any production site intended for tabletting.

### BRIEF DESCRIPTION OF THE FIGURES

FIG. 1 shows a graph representing exemplary results of the dissolution study in the below examples.

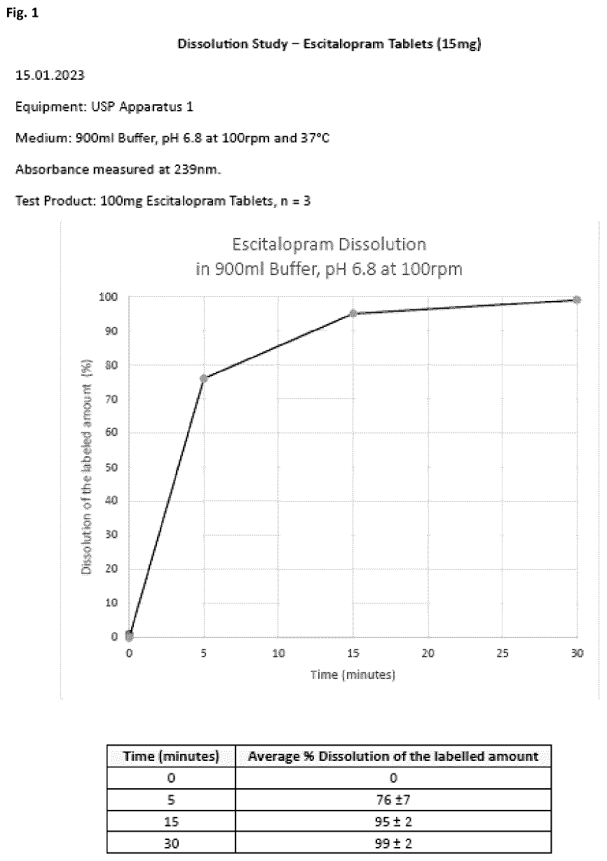

### EXAMPLES

### Example 1—Example Preparation of Escitalopram Film-Coated Tablets

Step 1. Excipients, if any or such as those of Tables 1 to 6 (the amounts being scaled according to the desired batch size), are passed through an appropriate screen. For the avoidance of doubt, a screen in this context refers to a sieve, preferably sieves with a pore size of 250 μm-710 μm.
Step 2. All excipients are blended with the API in a blender.
   For the avoidance of doubt, in view of the below tables it is apparent that Citalopram Oxalate or Escitalopram Oxalate is added; it can be added in Step 1 or 2.
Step 3. Lubricants are generally added last and blended.
Step 4. The final blend is compressed into tablets.
Step 5. A film-coating can be added onto the tablets.

The methods of preparation may include but are not limited to the direct compression and wet granulation methods.

### Example Compositions

TABLE 1
Composition Example 1

| Component | Composition (%) |
|---|---|
| Escitalopram Oxalate | 98.5 |
| Croscarmellose Sodium | 0.5 |
| Film-coating | 1.0 |
| Total | 100.0 |

TABLE 2
Composition Example 2

| Component | Composition (%) |
|---|---|
| Escitalopram Oxalate | 60.1 |
| Croscarmellose Sodium | 36.9 |
| Film-Coating | 3.0 |
| Total | 100.0 |

TABLE 3
Composition Example 3

| Component | Composition (%) |
|---|---|
| Escitalopram Oxalate | 75.0 |
| Microcrystalline Cellulose | 18.0 |
| Croscarmellose Sodium | 5.0 |
| Magnesium Stearate | 2.0 |
| Total | 100.0 |

TABLE 4
Composition Example 4

| Component | Composition (%) |
|---|---|
| Escitalopram Oxalate* | 25.1 |
| Croscarmellose Sodium | 1.0 |
| Microcrystalline Cellulose | 69.9 |
| Magnesium Stearate | 1.0 |
| Film-Coating | 3.0 |
| Total | 100.0 |

*Median particle size >40 μm

The median particle size is in particular determined using a Retsch analytical woven wire mesh sieve with a mesh size of 40 μm.

TABLE 5
Composition Example 5

| Component | Composition (%) |
|---|---|
| Escitalopram Oxalate | 100.0 |
| Total | 100.0 |

### Example 2—Example Tablet Weights Form Different Compositions

TABLE 6
Overview of example tablet weights

| Composition Example | Example Dosage | Example Total Weight |
|---|---|---|
| Composition Example 1 | 20 mg Tablet | 25.7 mg |
| Composition Example 2 | 5 mg Tablet | 10.6 mg |
| Composition Example 2 | 20 mg Tablet | 42.5 mg |
| Composition Example 3 | 20 mg Tablet | 34.1 mg |
| Composition Example 5 | 20 mg Tablet | 25.6 mg |

### Example 3—Analytical Results

TABLE 7
Disintegration Time & Resistance to Crushing

| Example | Disintegration Time | Resistance to Crushing |
|---|---|---|
| Composition | (Minutes) | (N) |
| 1 | <2 min | <20N |
| 2 | <2 min | <20N |
| 3 | <2 min | <20N |
| 4 | <2 min | <20N |
| 5 | <5 min | <10N |

A dissolution study was conducted on tablets of Example 3 in 900 ml of pH 6.8 buffer at 37° C., USP Apparatus 1 set to 100 rpm. The resultant dissolution curve demonstrates that the tablet rapidly dissolved with complete dissolution within 30 minutes.

The suitability of the quality of the immediate-release oral pharmaceutical form made under the invention is met, as it fulfils the requirements of the USP monograph for Escitalopram Tablets.

Certain embodiments of the invention are enumerated in the following:
1. A pharmaceutical composition of an immediate-release oral pharmaceutical form which comprises by weight of ≥60.1% Escitalopram Oxalate or ≥25.1% Escitalopram Oxalate (at a median particle size of more than 40 μm), and from 0% to 74.9% additional pharmaceutical excipient. More preferably it comprises by weigh of ≥70.1% Escitalopram Oxalate or ≥30.1% Escitalopram Oxalate (at a median particle size of more than 40 μm) and even more preferably it comprises by weight of ≥75.1% Escitalopram Oxalate or ≥60.1% Escitalopram Oxalate (at a median particle size of more than 40 μm).
2. The Escitalopram Oxalate of item 1. entails also the other pharmaceutically acceptable salts of Escitalopram.
3. The Escitalopram Oxalate of item 1. entails also the racemate (Citalopram) and pharmaceutically acceptable salts thereof.
4. The pharmaceutical excipient of item 1. consists of, but is not limited to, one or more disintegrant, lubricant, filler and glidant.
5. The disintegrant of item 4. consists of, but is not limited to microcrystalline cellulose, sodium alginate, Croscarmellose Sodium, croscarmellose sodium, crospovidone and any “super-disintegrants”.
6. The filler of item 4. consists of, but is not limited to cellulose, lactose, mannitol, starch, sucrose, and calcium phosphate.
7. The lubricant of item 4. consists of, but is not limited to calcium stearate, magnesium stearate, stearic acid, sodium lauryl sulphate, and magnesium lauryl sulphate.
8. The glidant of item 4. consists of, but is not limited to silica derivatives, talc, and corn starch.
9. The immediate-release oral pharmaceutical form of item 1. entails the tablet form, the film-coated tablet form and capsule form but is not limited to other types of immediate-release pharmaceutical forms. In the case of film-coated tablet form and capsule form, the percentage weight of Escitalopram or racemate (Citalopram) refers to the weight of the API in its entirety of the pharmaceutical form (which includes for example the film-coating or capsule shell).
10. The composition of item 1. is for the dosage range of 1.25 mg to 40 mg Escitalopram or racemate (Citalopram).

Further embodiments of the invention are enumerated in the following:
1. An immediate-release oral pharmaceutical composition comprising by weight of
   a) ≥60.1% Citalopram Oxalate or Escitalopram Oxalate or
   b) ≥25.1% Citalopram Oxalate or Escitalopram Oxalate at a median particle size of more than 40 μm, and
   one or more additional pharmaceutical excipients of from 0% to 74.9% by weight.

In preferred embodiments, the immediate-release oral pharmaceutical composition does not comprises further active agents, other than Citalopram Oxalate or Escitalopram Oxalate, meaning that it comprises e.g. by weight of ≥60.1% (or another percentage or a specific dosage, etc., as detailed herein, mutatis mutandis) of an active agent, wherein said active agent is Citalopram Oxalate or Escitalopram Oxalate.
2. Immediate-release oral pharmaceutical composition according to item 1, wherein
   a) the amount of Citalopram Oxalate or Escitalopram Oxalate by weight is ≥70.1%, preferably ≥75.1%
   or
   b) the amount of Citalopram Oxalate or Escitalopram Oxalate at a median particle size of more than 40 μm by weight is ≥30.1%, preferably ≥60.1%.

In another embodiment, the amount of Citalopram Oxalate or Escitalopram Oxalate in the immediate-release oral pharmaceutical composition is ≥75%, more preferably ≥98.5%, more preferably 100% by weight.
3. The Immediate-release oral pharmaceutical composition according to any one of items 1 or 2, wherein the additional pharmaceutical excipient comprises one or more components selected from the group comprising disintegrant, lubricant, filler and glidant.
4. The Immediate-release oral pharmaceutical composition according to item 3, wherein the disintegrant is one or more disintegrants selected from the group comprising microcrystalline cellulose, sodium croscarmellose, carboxymethyl cellulose, alginic acid, sodium alginate and their derivatives, sodium starch glycolate (such as Glycolys®, Explotab®, Vivastar® P), croscarmellose sodium (such as Ac Di Sol®, Solutab®, Vivasol®), crospovidone (such as Kollidon®, Kollicoat®) and other super-disintegrants, as well as cellulose, lactose, mannitol, starch and sucrose.
5. The immediate-release oral pharmaceutical composition according to any one of item 3 or 4, wherein the filler is one or more fillers selected from the group comprising cellulose, lactose, mannitol, starch, sucrose, and calcium phosphate, calcium carbonate, maltodextrin, sorbitol and dextrin.

It is known to the skilled person that certain excipients used as fillers can also act as disintegrant, such as cellulose, lactose, mannitol, starch and sucrose. In embodiments where one of these is included as disintegrant, they are not additionally used as fillers, in other words, their inclusion as “disintegrant” or “filler” in the embodiments of the present invention is mutually exclusive.
6. The immediate-release oral pharmaceutical composition according to any one of items 3 to 5, wherein the lubricant is one or more lubricants selected from the group comprising calcium stearate, magnesium stearate, stearic acid, sodium lauryl sulphate, and magnesium lauryl sulphate.
7. The immediate-release oral pharmaceutical composition according to any one of items 3 to 6, wherein the glidant is one or more glidants selected from the group comprising silica derivatives, such as silicone dioxide, talc, and corn starch.
8. The immediate-release oral pharmaceutical composition of any one of items 1 to 7, which is in tablet form or in a film-coated tablet form, wherein in said film-coated tablet form the percentage weight refers to the pharmaceutical composition in its entirety, including the film-coating weight of the tablet core.
9. The immediate-release oral pharmaceutical composition of any one of items 1 to 8, wherein Citalopram Oxalate or Escitalopram Oxalate is comprised in a dosage range of 1.25 mg to 40 mg, particularly 1.25 mg to 20 mg, or 1.25 mg to 5 mg, more particularly 5 mg to 20 mg.
10. A method of preparing a tablet comprising a composition according to any one of items 1 to 9, the method comprising
   a) passing the one or more pharmaceutical excipients through a screen,
   b) blending said one or more pharmaceutical excipients,
   c) optionally adding lubricant to the blend of b), and blend,
   d) compressing the blend obtained from c) into tablets, and
   e) optionally adding a film coating to the tablets,
   wherein Citalopram Oxalate or Escitalopram Oxalate is added either in step a) or between steps b) and c), and
   wherein the amounts by weight and dosage range of said excipients and of Citalopram Oxalate or Escitalopram Oxalate in the resulting tablet are as defined in the preceding items.

1. An immediate-release oral pharmaceutical composition comprising by weight of
a) ≥60.1% Citalopram Oxalate or Escitalopram Oxalate or
b) ≥25.1% Citalopram Oxalate or Escitalopram Oxalate at a median particle size of more than 40 μm, and
one or more additional pharmaceutical excipients of from 0% to 74.9% by weight.

2. The immediate-release oral pharmaceutical composition according to claim 1, wherein
a) the amount of Citalopram Oxalate or Escitalopram Oxalate by weight is ≥70.1%,
or
b) the amount of Citalopram Oxalate or Escitalopram Oxalate at a median particle size of more than 40 μm by weight is ≥30.1.

3. The immediate-release oral pharmaceutical composition according to claim 1, wherein the additional pharmaceutical excipient comprises one or more components selected disintegrants, lubricants, fillers, and glidants.

4. The immediate-release oral pharmaceutical composition according to claim 3, wherein the additional pharmaceutical excipient is one or more disintegrants selected from microcrystalline cellulose, sodium croscarmellose, carboxymethyl cellulose, alginic acid, sodium alginate and their derivatives, sodium starch glycolate (such as Glycolys®, Explotab®, Vivastar® P), croscarmellose sodium (such as Ac Di Sol®, Solutab®, Vivasol®), crospovidone (such as Kollidon®, Kollicoat®) and other super-disintegrants, as well as cellulose, lactose, mannitol, starch, and sucrose.

5. The immediate-release oral pharmaceutical composition according to claim 3, wherein the additional pharmaceutical excipient is one or more fillers selected from cellulose, lactose, mannitol, starch, sucrose, and calcium phosphate, calcium carbonate, maltodextrin, sorbitol, and dextrin.

6. The immediate-release oral pharmaceutical composition according to claim 3, wherein the additional pharmaceutical excipient is one or more lubricants selected from calcium stearate, magnesium stearate, stearic acid, sodium lauryl sulphate, and magnesium lauryl sulphate.

7. The immediate-release oral pharmaceutical composition according to claim 3, wherein the additional pharmaceutical excipient is one or more glidants selected from the group comprising silica derivatives, such as silicone dioxide, talc, and corn starch.

8. The immediate-release oral pharmaceutical composition of claim 1, which is in tablet form or in a film-coated tablet form, wherein in said film-coated tablet form the percentage weight refers to the pharmaceutical composition in its entirety, including the film-coating weight of the tablet core.

9. The immediate-release oral pharmaceutical composition of claim 1, wherein Citalopram Oxalate or Escitalopram Oxalate is comprised in a dosage range of 1.25 mg to 40 mg.

10. A method of preparing a tablet comprising a composition according to claim 1, the method comprising
a) passing the one or more pharmaceutical excipients through a screen,
b) blending said one or more pharmaceutical excipients,
c) optionally adding lubricant to the blend of b), and blend,
d) compressing the blend obtained from b) or c) into tablets, and
e) optionally adding a film coating to the tablets,
wherein Citalopram Oxalate or Escitalopram Oxalate is added either in step a) or between steps b) and c), and
wherein the amounts by weight and dosage range of said excipients and of Citalopram Oxalate or Escitalopram Oxalate in the resulting tablet are as defined in claim 1.

11. Immediate-release oral pharmaceutical composition according to claim 1, wherein
a) the amount of Citalopram Oxalate or Escitalopram Oxalate by weight is ≥75.1%,
or
b) the amount of Citalopram Oxalate or Escitalopram Oxalate at a median particle size of more than 40 μm by weight is ≥60.1%.

12. The immediate-release oral pharmaceutical composition of claim 1, wherein Citalopram Oxalate or Escitalopram Oxalate is comprised in a dosage range of 1.25 mg to 20 mg.

13. The immediate-release oral pharmaceutical composition of claim 1, wherein Citalopram Oxalate or Escitalopram Oxalate is comprised in a dosage range of 1.25 mg to 5 mg.

14. The immediate-release oral pharmaceutical composition of claim 1, wherein Citalopram Oxalate or Escitalopram Oxalate is comprised in a dosage range of 5 mg to 20 mg.
